(19) United States
(12) Patent Application Publication
(10) Pub. No.: US 2023/0009630 A1
(43) Pub. Date: Jan. 12, 2023

(54) IMPROVEMENTS IN WEIGHT CONTROL

(71) Applicant: JUDD HEALTH LIMITED, Oxfordshire (GB)

(72) Inventors: Dominic CHANDLER, Oxfordshire (GB); Chris HUNTLY-GORDON, Oxfordshire (GB)

(21) Appl. No.: 17/781,871

(22) PCT Filed: Dec. 2, 2020
(86) PCT No.: PCT/GB2020/053088

§ 371 Date: Jun. 2, 2022

(30) Foreign Application Priority Data
Dec. 2, 2019 (GB) 1917595.9

(51) Int. Cl. A61K 9/00; A61K 31/11; A61K 47/10; A61K 47/14

(52) U.S. Cl. CPC A61K 9/007; A61K 9/0043; A61K 31/11; A61K 47/10; A61K 47/14

(57) ABSTRACT

Food scent substance for use in the treatment of obesity, wherein food scent from the food scent substance is inhaled from a device comprising the food scent substance.

### FIELD OF THE INVENTION

The present invention relates to devices, methods and substances for weight control.

### BACKGROUND OF THE INVENTION

In today's society, many people are overweight or obese and this is usually linked with their diet. People experience cravings for unhealthy foods (e.g. foods that have a high sugar, fat or salt content), which can lead to them becoming overweight if this is not counterbalanced by other lifestyle choices, such as exercise.

How does your brain process signals?

With advancements in electroencephalogram (EEG), positron emission tomography (PET), and functional magnetic resonance imaging (fMRI) researchers have now begun to gain a better understanding of which parts of the brain are responsible for processing stimuli from different sensory modalities.

Rather than each structure having a single function, it is now understood that there are multisensory regions in the brain that act as convergence zones where inputs from different sensory modalities combine, interact, and influence each other_{4,5}.

This means that sensory signals from one source can influence how a signal from a different source is interpreted. For example, it has been proven that solutions that smell of strawberry are perceived to smell stronger when they are coloured red₆. Demonstrating how a visual cue can cause changes to your perceived sense of smell. This is called the cross-modal sensory effect.

How does this affect your cravings?

Amongst the five senses, the olfactory and gustatory systems are especially interconnected_{7,5,9}. Together with the trigeminal system, they make up the chemosensory system, which is responsible for flavour perception₁.

When you receive an olfactory stimulus, signals are sent from the olfactory bulb to the piriform cortex and then the orbitofrontal cortex. It has recently been discovered that the orbitofrontal cortex is one of the multisensory regions, predominantly for taste and olfactory signals_{1,7,8}.

One of the jobs of the orbitofrontal cortex is to determine the reward value₉. Once a stimulus with a high reward value is identified, the brain's reward circuitry, primarily composed of the dopamine system, is activated_{10,11}.

The key here is that whilst the orbitofrontal cortex discriminates between stimuli on the basis of valence as well as the intensity of the reward value_{14, 15}, it does not discriminate among the sensory modalities in which the stimuli are encoded₁₆.

This is what is referred to as the cross-modal sensory compensation effect; where stimuli from one sense can satisfy the desire related to another sense₃. At least partial olfactory, as well as taste, sensory-specific satiety does not require food to enter the gastrointestinal system and does not depend on the ingestion of calories₂. The sense of smell is particularly suited to this as the body can adjust its responses rapidly to differing odour environments₁₈.

What actually is the reward?

Rewards are, in the simplest terms, the environmental incentives we tend to approach. More precisely, they are the environmental incentives we turn to after having previously contacted them. It is the return to a reward previously experienced that is the essence of habit and addiction₁₁. Therefore, what we tend to designate as the receipt of reward might more accurately be designated as simply a more proximal predictor of reward₁₁. This is because the dopamine systems are aroused more by the sensory cues that predict receipt of the reward than they are by the actual receipt of the reward₁₁.

Rewards have no dedicated receptors. Value is not a physical property but determined by brain activity that interprets the potential effect of a reward on survival and reproduction. Thus rewards are internal to the brain and based entirely on brain function₁₉.

Esthetic (and monetary) value is entirely determined by the subjective value assigned by our brain following the sensory processing. Although we process great taste or smell as sensory events, we appreciate them as motivating and pleasing due to our subjective valuation₁₉.

External visual, somatic, auditory, olfactory, and gustatory stimuli predicting original, unconditioned rewards become conditioned rewards through Pavlovian conditioning₁₉. The brain was not made for enjoying a great meal; it was made for getting the best food for survival.

For eating, most people assume that taste is the reward, however, it appears to just be a predictor of the reward. A 1959 study which gave animals neutrally flavoured foods, each accompanied by an intragastric glucose load found that the animals learned flavour preferences that were proportional to the associated glucose load₂₀. Demonstrating that the stamping of food-rewarded memories appears to depend critically on the postingestinal consequences of food₁₀. And that the reinforcement process begins some significant time after the taste of the food; the taste of food is, like the smell and sight of food and the sounds that precede the delivery of food, a predictor of reward and not the primary reward itself (cross-modal sensory compensation effect)₁₁.

Another study involved mice with knocked out sweet taste receptors, who still learnt to approach and choose sucrose, suggesting that the calories and the resulting blood sugar increase constitute the unconditioned reward effect instead of or in addition to taste₂₁.

The key point here is that people who suffer from obesity have been conditioned by the predictors of rewards (in this case, the sight and particularly the smell of indulgent foods) to regularly eat foods with excessive sugar, salt, and fat.

The references identified in subscript in the preceding paragraphs are listed below.
1. Lundstrom, Johan N., Boesvelt, Sanne, Albrecht, Jessica (2011), “Central Processing of the Chemical Senses: An Overview,” ACS Chemical Neuroscience, 2 (1), 5-16.
2. Rolls, Edmund T., Rolls, J. H. (1997). “Olfactory Sensory-Specific Satiety in Humans,” Physiology & Behavior, 61 (3) 461-73.
3. Biswas, Dipayan, Labrecque, Lauren, Lehmann, Donald, Markos, Ereni (2014), “Making Choices While Smelling, Tasting, and Listening: The Role of Sensory (Dis)similarity When Sequentially Sampling Products,” Journal of Marketing, 78 (1), 112-26.
4. Driver, Jon, Noesselt, Toemme (2008), “Multisensory Interplay Reveals Crossmodal Influences on “Sensory-Specific” Brain Regions, Neural Responses, and Judgments,” Neuron, 57 (1), 11-23.
5. Van Atteveldt, Nienke, Murray, Micah M., Thut, Gregor, Schroeder, C. E. (2014). “Multisensory Integration: Flexible Use of General Operations,” Neuron, 81 (6), 1240-53.
6. Zellner, Debra A., and Mary A. Kautz (1990), “Cold Affects Perceived Odor Intensity,” journal of Experimental Psychology: Human Perception and Performance, 16 (2), 391-97.
7. De Araujo, Ivan E. T., Rolls, Edmund T., Kringelbach, Morten L., McGlone, Francis, Phillips, Nicola (2003), “Taste-Olfactory Convergence, and the Representation of the Pleasantness of Flavour, in the Human Brain,” European journal of Neuroscience, 18 (7), 2059-68.
8. Gagnon, Lea, Vestergaard, Martin, Madsen, Kristoffer, Karstensen, Helena G., Siebner, Hartwig, Tommerup, Niels. (2014), “Neural Correlates of Taste Perception in Congenital Olfactory lmpairrnent,” Neuropsychologica, 62,297-305.
9. Rolls, Edmund T. (2008), “Functions of the Orbitofrontal and Pregenual Cingulate Cortex in Taste, Olfaction, Appetite, and Emotion,” Acta Physiologica Hungarica, 95 (2), 131-64.
10. Carnerer, Colin, Loewenstein, George, Preiec, Drazen (2005), “Neuroeconomics: How Neuroscience Can Inform Economics,” Journal of Economic Literature, 43 (March), 9-64,
11. Wise, Roy A. (2002), “Brain Reward Circuitry: Insights from Unsensed Incentives,” Neuron, 36 (2), 229-40.
12. Rots, Edmund T. (2011), “Taste, Olfactory and Food Texture Reward Processing in the Brain and Obesity,” International Journal of Obesity, 35 (4), 550-61.
13. Kringelbach, M. L., O'Doherty, J., Rolls, E. T., Andrews, C. (2003), “Activation of Human Orbitofrontal Cortex to a Liquid Food Stimulus is Correlated with its Subjective Pleasantness,” Cerebral Cortex, 13 (10), 1064-71.
14. HoHerman, Jeffrey R., Tremblay, Leon, Schultz, Wolfram (1998), “Influence of Reward Expectation on Behavior-Related Neuronal Activity in Primate Striatum,” Journal of Neurophysiology, 80 (2), 947-63.
15. Tremblay, Leon, Schultz, Wolfram (1999), “Relative Reward Preference in Primate Orbitofrontal Cortex,” Nature, 398,704-08.
16. Schultz, Wolfram (2002), “Getting Formal with Dopamine and Reward.” Neuron, 36 (2), 241-63.
17. Biswas, D., & Szocs, C. (2019). The Smell of Healthy Choices: Cross-Modal Sensory Compensation Effects of Ambient Scent on Food Purchases. Journal of Marketing Research.
18. Boesveldt, Sanne, de Graaf, Kees (2017), “The Differential Role of Smell and Taste For Eating Behaviour,” Perception, 46 (3-4), 307-319.
19. Schultz, Wolfram. (2015). Neuronal Reward and Decision Signals: From Theories to Data. Physiological reviews. 95.853-951.
20. Le Magnen, J. (1959), Effects des administrations postprandiales de glucose dans l'établissement des appetits. C R Seances Soc Biol Fil., 153,212-5.
21. De Araujo I E, Oliveira-Main A J, Sotnikova T D, Gainetdinov R R, Caron M C, Nicolelis M A L, Simon S A (2008) “Food reward in the absence of taste receptor signalling,” Neuron 57: 930-941.

### Effect of Indulgent/Nonindulgent Scents on Food Choices

In Biswas, D., & Szocs, C. (2019). The Smell of Healthy Choices: Cross-Modal Sensory Compensation Effects of Ambient Scent on Food Purchases. Journal of Marketing Research, three studies conducted at a supermarket and a middle school cafeteria, and four studies conducted in the lab, demonstrated that extended exposure (of more than two minutes) to an indulgent food-related ambient scent (vs. a nonindulgent scent or no scent) leads to reduced purchase of unhealthy foods. The authors proposed that this occurs because scents related to an indulgent food satisfy the reward circuitry in the brain, which in turn reduces the urge for actual consumption of indulgent foods. Examples of indulgent scents were pizza and cookie and examples of nonindulgent scents were apple and strawberry.

Participants were exposed to the indulgent and nonindulgent scents passively using commercial grade scent nebulizers. The duration of exposure was shown to be important. Where the duration of exposure to the indulgent scent was more than two minutes, this reduced participants' preference for an unhealthy option. However, when the duration of exposure to the indulgent scent was less than 30 seconds, the trend was reversed and this increased participants' preference for an unhealthy option.

There is therefore evidence that prolonged exposure (e.g. at least two minutes) to indulgent scents results in healthier food choices, which could help with weight control. However, providing such prolonged exposure (e.g. using scent nebulizers or diffusers, which are intended to produce a fine mist and to diffuse throughout an entire room) is challenging, overly intrusive and problematic.

The invention provides a solution to this and other problems.

### SUMMARY OF THE INVENTION

In accordance with the present invention, there is provided food scent substance for use in the treatment of obesity, wherein food scent from the food scent substance is inhaled (e.g. inhaled nasally) from a device comprising the food scent substance.

It has been shown that prolonged exposure to some food scents (e.g. indulgent food scents, such as vanilla, pizza and cookie) suppresses the craving to consume indulgent (e.g. sugary, fatty, salty) food. Such prolonged exposure is challenging to provide e.g. using scent nebulizers, diffusers and similar methods which are intended to produce a fine mist and to diffuse throughout an entire room because such methods are overly intrusive and uncomfortable. The present invention provides a transportable device, for example an inhaler, from which a user may conveniently, comfortably and discreetly inhale a food scent for a sufficient duration to suppress food cravings, thereby helping with weight control and treating obesity. People who suffer from obesity have been conditioned by the predictors of rewards (in this case, the sight and particularly the smell of indulgent foods) to regularly eat foods with excessive sugar, salt, and fat. The present invention uses the cross-modal sensory compensation effect to break this cycle; using the olfactory system to trigger the reward system and stop the craving that was only created by the anticipation of sugar, salt, or fat rather than the actual need for it.

The device may be at least partially inserted into a user's nostril whilst the food scent is being inhaled. The food scent substance may be inhaled for a sufficient duration (which may be done intermittently) to suppress food cravings, for example for at least 30 seconds, for example at least one minute, for example at least 90 seconds, for example at least two minutes.

The device may comprise a device body, and at least a portion of the device body may have a substantially circumferential cross-sectional shape. This shape is convenient for a user to transport (e.g. in a bag or pocket), convenient for a user to hold during use, and is a convenient shape from which to inhale the food scent substance nasally (e.g. by at least partially inserting the device into a nostril). The device body may have a first end that is inserted into a user's nostril and an opposing second end, wherein the first end may have a rounded shape, for example a hemispherical shape. This rounded shape is a comfortable and convenient shape for at least partial insertion into a nostril.

The food scent may be an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread. An indulgent food scent can be sweet or savoury and is a scent of a food that would be considered a treat, for example unhealthy foods, such as foods with a high fat, sugar and/or salt content that may be detrimental to health if consumed regularly, or in excess, or regularly in excess. For example, they may be foods that may cause a person to gain weight if consumed regularly, or in excess, or regularly in excess without counterbalancing their consumption with sufficient exercise. Indulgent foods may include “junk food”. The indulgent food scent may also be a scent often found in indulgent foods, such as vanilla. The opposite of an indulgent food scent would be a nonindulgent food scent, such as a vegetable scent or a scent associated with healthy or nutritious foods.

The skilled person will realise that any commercially available indulgent food scent could be used as long as it satisfies safety/regulatory requirements. Indulgent food scent compounds can comprise certain functional groups that are characteristic of compounds with olfactory properties. These functional groups include alcohols, aldehydes, ethers, esters, ketones, lactones and thiols. Such alternative indulgent food scent compounds would be well understood by the skilled person, and it would be readily conceived to use any of these such indulgent food scents.

One example of an indulgent food scent is vanilla, in which case the food scent may contain vanilla flavour. The flavour of vanilla results largely from the organic compound vanillin. Vanillin is a phenolic aldehyde. Its functional groups include aldehyde, hydroxyl, and ether. It is the primary component of the extract of the vanilla bean. The chemical structure of vanillin, whose chemical formula is 4-hydroxy-3-methoxybenzaldehyde, is shown below:

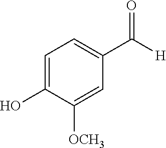 [SMILES: COc1cc(C=O)ccc1O]

Vanillin may be provided as a natural extract from vanilla pods. This natural extract may also contain other chemical compounds which include acetaldehyde, acetic acid, furfural, hexanoic acid, 4-hydroxybenzaldehyde, eugenol, methyl cinnamate, and isobutyric acid, which all may contribute to overall scent of vanilla.

Vanillin may alternatively be provided alone as the sole contributor to the vanillin scent. This vanillin may be isolated from natural extract or may be chemically synthesised, such as by standard industry methods that are known in the art. This vanillin may be provided in a solution, such as in an ethanolic solution.

The scent of vanilla may also be provided by ethylvanillin, a chemical compound that is related to vanillin. Ethylvanillin is approximately three times as potent as vanillin. The chemical structure of ethylvanillin, 3-ethoxy-4-hydroxybenzaldehyde, is shown below:

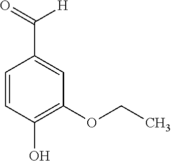

As used herein, the term vanillin may be used interchangeably with ethylvanillin.

As well as the scented substance itself, the food scent substance may contain additional ingredients. For example, carrier substances could be included to carry the food scent. These carrier substances may perform any function that enables the food scent to be carried, such as by modifying the volatility of the food scent. Such carrier substances would be well known to the skilled person. Alternatively, other ingredients may be included in the food scent substance such as emulsifying agents, stabilizing agents, binders, lubricants, buffers, diluents, preservatives and humectants. Suitable ingredients to perform these functions would be well known to the skilled person.

In one example of a vanilla food scent substance, the breakdown of ingredients by mass is as follows:

Natural Flavouring substances (including vanillin): 20.38%

Propylene Glycol (Carrier): 16.16%

Triacetin (Carrier): 63.46%

A total mass of between 200 milligrams and 1 gram of the food scent substance may be used inside an inhaler, for example between 400 milligrams and 800 milligrams, for example between 500 milligrams and 700 milligrams, for example between 550 milligrams and 650 milligrams. In one example, a total mass of 620 milligrams of the food scent substance is used inside an inhaler.

The device may contain a substrate (e.g. cotton wool) and the food scent substance may be disposed on the substrate. Cotton wool is a cheap, convenient and effective way of retaining a food scent substance (e.g. in liquid form) on a substrate. The substrate may be contained within the device body. Alternatively, the food scent substance may be disposed on at least a portion of the interior and/or the exterior of the device.

The food scent substance may be provided as a liquid solution (aqueous or non-aqueous), an oil, a wax, a gel, a gas (e.g. a compressed gas), a solid, a powder or any other suitable form.

There is also provided a method comprising providing a device comprising a food scent substance; and inhaling food scent from the food scent substance (e.g. inhaling nasally).

It has been shown that prolonged exposure to some food scents (e.g. indulgent food scents, such as vanilla, pizza and cookie) suppresses the craving to consume indulgent (e.g. sugary, fatty, salty) food. Such prolonged exposure is challenging to provide e.g. using scent nebulizers, diffusers and similar methods which are intended to produce a fine mist and to diffuse throughout an entire room because such methods are overly intrusive and uncomfortable. The present invention provides a transportable device, for example an inhaler, from which a user may conveniently, comfortably and discreetly inhale a food scent for a sufficient duration to suppress food cravings, thereby helping with weight control and treating obesity.

The device may be at least partially inserted into a user's nostril whilst the food scent is being inhaled. The food scent substance may be inhaled for a sufficient duration (which may be done intermittently) to suppress food cravings, for example for at least 30 seconds, for example at least one minute, for example at least 90 seconds, for example at least two minutes.

The device may comprise a device body, and at least a portion of the device body may have a substantially circumferential cross-sectional shape. This shape is convenient for a user to transport (e.g. in a bag or pocket), convenient for a user to hold during use, and is a convenient shape from which to inhale the food scent substance nasally (e.g. by at least partially inserting the device into a nostril). The device body may have a first end that is inserted into a user's nostril and an opposing second end, wherein the first end may have a rounded shape, for example a hemispherical shape. This rounded shape is a comfortable and convenient shape for at least partial insertion into a nostril.

The food scent may be an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread. An indulgent food scent can be sweet or savoury and is a scent of a food that would be considered a treat, for example unhealthy foods, such as foods with a high fat, sugar and/or salt content that may be detrimental to health if consumed regularly, or in excess, or regularly in excess. For example, they may be foods that may cause a person to gain weight if consumed regularly, or in excess, or regularly in excess without counterbalancing their consumption with sufficient exercise. Indulgent foods may include “junk food”. The indulgent food scent may also be a scent often found in indulgent foods, such as vanilla. The opposite of an indulgent food scent would be a nonindulgent food scent, such as a vegetable scent or a scent associated with healthy or nutritious foods.

The skilled person will realise that any commercially available indulgent food scent could be used as long as it satisfies safety/regulatory requirements. Indulgent food scent compounds can comprise certain functional groups that are characteristic of compounds with olfactory properties. These functional groups include alcohols, aldehydes, ethers, esters, ketones, lactones and thiols. Such alternative indulgent food scent compounds would be well understood by the skilled person, and it would be readily conceived to use any of these such indulgent food scents.

One example of an indulgent food scent is vanilla, in which case the food scent may contain vanilla flavour. The flavour of vanilla results largely from the organic compound vanillin. Vanillin is a phenolic aldehyde. Its functional groups include aldehyde, hydroxyl, and ether. It is the primary component of the extract of the vanilla bean. The chemical structure of vanillin, whose chemical formula is 4-hydroxy-3-methoxybenzaldehyde, is shown below:

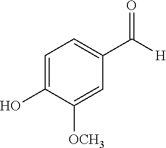 [SMILES: COc1cc(C=O)ccc1O]

Vanillin may be provided as a natural extract from vanilla pods. This natural extract may also contain other chemical compounds which include acetaldehyde, acetic acid, furfural, hexanoic acid, 4-hydroxybenzaldehyde, eugenol, methyl cinnamate, and isobutyric acid, which all may contribute to overall scent of vanilla.

Vanillin may alternatively be provided alone as the sole contributor to the vanillin scent. This vanillin may be isolated from natural extract or may be chemically synthesised, such as by standard industry methods that are known in the art. This vanillin may be provided in a solution, such as in an ethanolic solution.

The scent of vanilla may also be provided by ethylvanillin, a chemical compound that is related to vanillin. Ethylvanillin is approximately three times as potent as vanillin. The chemical structure of ethylvanillin, 3-ethoxy-4-hydroxybenzaldehyde, is shown below:

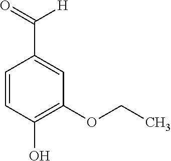

As used herein, the term vanillin may be used interchangeably with ethylvanillin.

As well as the scented substance itself, the food scent substance may contain additional ingredients.

For example, carrier substances could be included to carry the food scent. These carrier substances may perform any function that enables the food scent to be carried, such as by modifying the volatility of the food scent. Such carrier substances would be well known to the skilled person. Alternatively, other ingredients may be included in the food scent substance such as emulsifying agents, stabilizing agents, binders, lubricants, buffers, diluents, preservatives and humectants. Suitable ingredients to perform these functions would be well known to the skilled person.

In one example of a vanilla food scent substance, the breakdown of ingredients by mass is as follows:

Natural Flavouring substances (including vanillin): 20.38%

Propylene Glycol (Carrier): 16.16%

Triacetin (Carrier): 63.46%

A total mass of between 200 milligrams and 1 gram of the food scent substance may be used inside an inhaler, for example between 400 milligrams and 800 milligrams, for example between 500 milligrams and 700 milligrams, for example between 550 milligrams and 650 milligrams. In one example, a total mass of 620 milligrams of the food scent substance is used inside an inhaler.

The device may contain a substrate (e.g. cotton wool) and the food scent substance may be disposed on the substrate. Cotton wool is a cheap, convenient and effective way of retaining a food scent substance (e.g. in liquid form) on a substrate. The substrate may be contained within the device body. Alternatively, the food scent substance may be disposed on at least a portion of the interior and/or the exterior of the device.

The food scent substance may be provided as a liquid solution (aqueous or non-aqueous), an oil, a wax, a gel, a gas (e.g. a compressed gas), a solid, a powder or any other suitable form.

There is also provided an inhaler comprising a food scent substance and means for inhaling (e.g. nasally inhaling) a food scent from the food scent substance. The means for inhaling the food scent may, for example, be an aperture through which the food scent can exit the inhaler for inhalation.

It has been shown that prolonged exposure to some food scents (e.g. indulgent food scents, such as vanilla, pizza and cookie) suppresses the craving to consume indulgent (e.g. sugary, fatty, salty) food. Such prolonged exposure is challenging to provide e.g. using scent nebulizers, diffusers and similar methods which are intended to produce a fine mist and to diffuse throughout an entire room because such methods are overly intrusive and uncomfortable. The present invention provides a transportable device, for example an inhaler, from which a user may conveniently, comfortably and discreetly inhale a food scent for a sufficient duration to suppress food cravings, thereby helping with weight control and treating obesity.

The inhaler may be at least partially inserted into a user's nostril whilst the food scent is being inhaled. The food scent substance may be inhaled for a sufficient duration (which may be done intermittently) to suppress food cravings, for example for at least 30 seconds, for example at least one minute, for example at least 90 seconds, for example at least two minutes.

The inhaler may comprise a body, and at least a portion of the body may have a substantially circumferential cross-sectional shape. This shape is convenient for a user to transport (e.g. in a bag or pocket), convenient for a user to hold during use, and is a convenient shape from which to inhale the food scent substance nasally (e.g. by at least partially inserting the device into a nostril). The inhaler body may have a first end that is configured to be inserted into a user's nostril and an opposing second end, wherein the first end may have a rounded shape, for example a hemispherical shape. This rounded shape is a comfortable and convenient shape for at least partial insertion into a nostril.

The food scent may be an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread. An indulgent food scent can be sweet or savoury and is a scent of a food that would be considered a treat, for example unhealthy foods, such as foods with a high fat, sugar and/or salt content that may be detrimental to health if consumed regularly, or in excess, or regularly in excess. For example, they may be foods that may cause a person to gain weight if consumed regularly, or in excess, or regularly in excess without counterbalancing their consumption with sufficient exercise. Indulgent foods may include “junk food”. The indulgent food scent may also be a scent often found in indulgent foods, such as vanilla. The opposite of an indulgent food scent would be a nonindulgent food scent, such as a vegetable scent or a scent associated with healthy or nutritious foods.

The skilled person will realise that any commercially available indulgent food scent could be used as long as it satisfies safety/regulatory requirements. Indulgent food scent compounds can comprise certain functional groups that are characteristic of compounds with olfactory properties. These functional groups include alcohols, aldehydes, ethers, esters, ketones, lactones and thiols. Such alternative indulgent food scent compounds would be well understood by the skilled person, and it would be readily conceived to use any of these such indulgent food scents.

One example of an indulgent food scent is vanilla, in which case the food scent may contain vanilla flavour. The flavour of vanilla results largely from the organic compound vanillin. Vanillin is a phenolic aldehyde. Its functional groups include aldehyde, hydroxyl, and ether. It is the primary component of the extract of the vanilla bean. The chemical structure of vanillin, whose chemical formula is 4-hydroxy-3-methoxybenzaldehyde, is shown below:

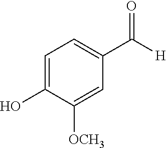 [SMILES: COc1cc(C=O)ccc1O]

Vanillin may be provided as a natural extract from vanilla pods. This natural extract may also contain other chemical compounds which include acetaldehyde, acetic acid, furfural, hexanoic acid, 4-hydroxybenzaldehyde, eugenol, methyl cinnamate, and isobutyric acid, which all may contribute to overall scent of vanilla.

Vanillin may alternatively be provided alone as the sole contributor to the vanillin scent. This vanillin may be isolated from natural extract or may be chemically synthesised, such as by standard industry methods that are known in the art. This vanillin may be provided in a solution, such as in an ethanolic solution.

The scent of vanilla may also be provided by ethylvanillin, a chemical compound that is related to vanillin. Ethylvanillin is approximately three times as potent as vanillin. The chemical structure of ethylvanillin, 3-ethoxy-4-hydroxybenzaldehyde, is shown below:

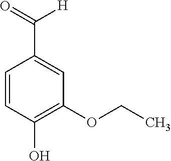

As used herein, the term vanillin may be used interchangeably with ethylvanillin.

As well as the scented substance itself, the food scent substance may contain additional ingredients. For example, carrier substances could be included to carry the food scent. These carrier substances may perform any function that enables the food scent to be carried, such as by modifying the volatility of the food scent. Such carrier substances would be well known to the skilled person. Alternatively, other ingredients may be included in the food scent substance such as emulsifying agents, stabilizing agents, binders, lubricants, buffers, diluents, preservatives and humectants. Suitable ingredients to perform these functions would be well known to the skilled person.

In one example of a vanilla food scent substance, the breakdown of ingredients by mass is as follows:

Natural Flavouring substances (including vanillin): 20.38%

Propylene Glycol (Carrier): 16.16%

Triacetin (Carrier): 63.46%

A total mass of between 200 milligrams and 1 gram of the food scent substance may be used inside an inhaler, for example between 400 milligrams and 800 milligrams, for example between 500 milligrams and 700 milligrams, for example between 550 milligrams and 650 milligrams. In one example, a total mass of 620 milligrams of the food scent substance is used inside an inhaler.

The device may contain a substrate (e.g. cotton wool) and the food scent substance may be disposed on the substrate. Cotton wool is a cheap, convenient and effective way of retaining a food scent substance (e.g. in liquid form) on a substrate. The substrate may be contained within the device body. Alternatively, the food scent substance may be disposed on at least a portion of the interior and/or the exterior of the device.

The food scent substance may be provided as a liquid solution (aqueous or non-aqueous), an oil, a wax, a gel, a gas (e.g. a compressed gas), a solid, a powder or any other suitable form.

Also provided is a use of a food scent substance in the manufacture of a medicament for the treatment of obesity, wherein food scent from the food scent substance is inhaled (e.g. inhaled nasally) from a device comprising the food scent substance.

Also provided is a method for treating obesity using a food scent substance, wherein food scent from the food scent substance is inhaled (e.g. inhaled nasally) from a device comprising the food scent substance.

### BRIEF DESCRIPTION OF THE DRAWINGS

The present invention will now be described with reference to the accompanying drawings, in which:

FIG. 1 is a perspective view of a device according to the invention in a closed configuration with a lid in place;

FIG. 2 is a perspective view of the device of FIG. 1 disassembled with the contents removed.

### DETAILED DESCRIPTION

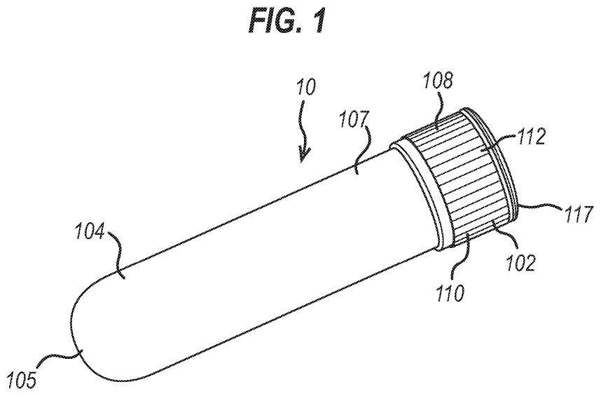

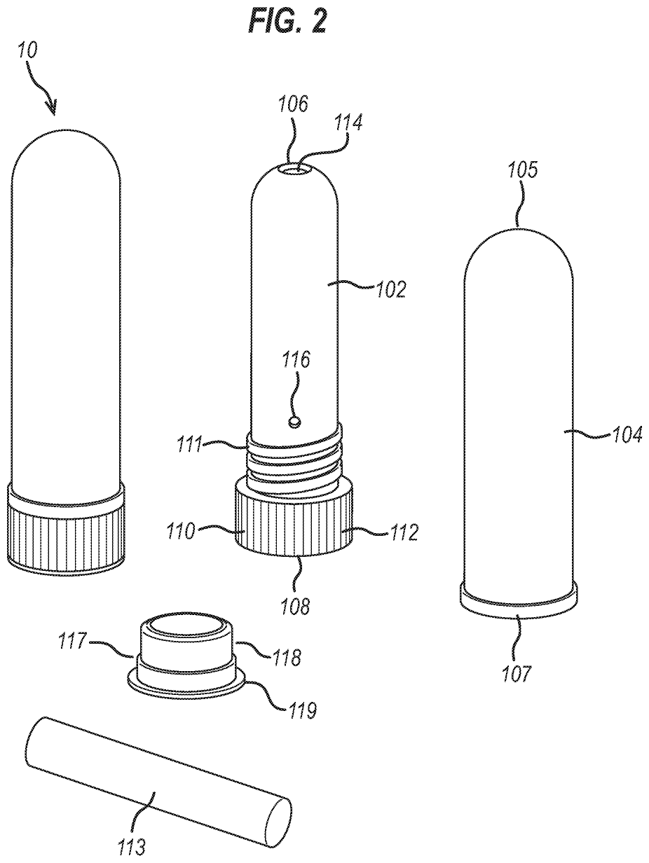

FIG. 1 shows a perspective view of a device (i.e. an inhaler) 10 according to the present invention. FIG. 2 shows a perspective view of the inhaler 10 of FIG. 1 disassembled with the contents removed.

The inhaler 10 comprises a body 102 and a lid 104. At least a portion of both the body 102 and the lid 104 have a substantially circular or circular cross-sectional shape. In other words, a portion of the body 102 and the lid 104 is substantially cylindrical or cylindrical. This shape is convenient for a user to transport (e.g. in a bag or pocket), convenient for a user to hold during use, and is a convenient shape from which to inhale the food scent substance nasally (e.g. by at least partially inserting the inhaler body 102 into a nostril).

The inhaler body 102 has a first end 106 that is configured to be inserted into a user's nostril and an opposing second end 108. It can be seen from FIG. 2 that the first end 106 has a rounded shape. In the embodiment shown in FIG. 2, the first end 106 has a hemispherical shape. This rounded shape is a comfortable and convenient shape for at least partial insertion into a nostril. In the embodiment of FIGS. 1 and 2, it can be seen that the lid 104 also has a first end 105 and a second end 107. Like the body 102, the first end 105 of the lid 104 also has a rounded e.g. hemispherical shape thereby ensuring a secure and snug fit between the first ends 106, 105 of the body 102 and the lid 104.

The second end 108 of the body 102 comprises a grip 110 that assists a user in removing the lid 104 from the body 102. The grip 110 comprises a plurality of ridges 112 extending around the circumference of the second end 108 of the body 102. The skilled person will understand that other gripping means could be used. Whilst not shown in the embodiment of FIG. 2, the second end 107 of the lid 104 may or may not also comprise a grip. In the embodiment shown in FIGS. 1 and 2, the body 102 and the lid 104 are attached to and detached from each other by screwing. This is achieved by screw threads 111 on the exterior of the body 102 and complementary screw threads (not shown) on the interior of the lid 104. The skilled person will understand that other suitable attachment means (e.g. snap-fit, interference fit) could equally be used.

FIG. 2 shows the inhaler 10 of FIG. 1 disassembled with the contents removed. The inhaler 10 comprises a food scent substance (not shown). The food scent substance may be provided as a liquid solution (aqueous or non-aqueous), an oil, a wax, a gel, a gas (e.g. a compressed gas), a solid, a powder or any other suitable form. In the embodiment of FIG. 2, the body 102 contains a substrate 113 in the form of cotton wool onto which the food scent substance is disposed.

Alternatively, the food scent substance could be disposed on at least a portion of the interior and/or the exterior of the inhaler 10, particularly the inhaler body 102, or at least a portion of the inhaler 10, particularly the inhaler body 102, may be pre-scented with a food scent. The body 102 of the inhaler 10 shown in FIG. 2 comprises at least one aperture 114 through which the food scent is inhaled. The body 102 shown in FIG. 2 also comprises at least one pinhole 116 to allow for the interior of the body 102 to be ventilated and to assist with air passage through the body 102 and, consequently, inhalation of the food scent. The inhaler 10 also comprises a base unit 117 that comprises a cylindrical portion 118 and an annular flange 119. The base unit 117 is configured to be removably attachable to the second end 108 of the body 102 (e.g. via a snap fit or interference fit) to support the substrate 113 and seal the second end 108 of the body 102. In alternative embodiments, there may be no base unit and the second end 108 of the body 102 may simply have a wall to seal the second end 108 of the body 102.

The food scent may be an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread. An indulgent food scent can be sweet or savoury and is a scent of a food that would be considered a treat, for example unhealthy foods, such as foods with a high fat, sugar and/or salt content that may be detrimental to health if consumed regularly, or in excess, or regularly in excess. For example, they may be foods that may cause a person to gain weight if consumed regularly, or in excess, or regularly in excess without counterbalancing their consumption with sufficient exercise. Indulgent foods may include “junk food”. The indulgent food scent may also be a scent often found in indulgent foods, such as vanilla. The opposite of an indulgent food scent would be a nonindulgent food scent, such as a vegetable scent or a scent associated with healthy or nutritious foods.

The skilled person will realise that any commercially available indulgent food scent could be used as long as it satisfies safety/regulatory requirements. Indulgent food scent compounds can comprise certain functional groups that are characteristic of compounds with olfactory properties. These functional groups include alcohols, aldehydes, ethers, esters, ketones, lactones and thiols. Such alternative indulgent food scent compounds would be well understood by the skilled person, and it would be readily conceived to use any of these such indulgent food scents.

One example of an indulgent food scent is vanilla, in which case the food scent may contain vanilla flavour. The flavour of vanilla results largely from the organic compound vanillin. Vanillin is a phenolic aldehyde. Its functional groups include aldehyde, hydroxyl, and ether. It is the primary component of the extract of the vanilla bean. The chemical structure of vanillin, whose chemical formula is 4-hydroxy-3-methoxybenzaldehyde, is shown below:

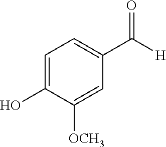 [SMILES: COc1cc(C=O)ccc1O]

Vanillin may be provided as a natural extract from vanilla pods. This natural extract may also contain other chemical compounds which include acetaldehyde, acetic acid, furfural, hexanoic acid, 4-hydroxybenzaldehyde, eugenol, methyl cinnamate, and isobutyric acid, which all may contribute to overall scent of vanilla.

Vanillin may alternatively be provided alone as the sole contributor to the vanillin scent. This vanillin may be isolated from natural extract or may be chemically synthesised, such as by standard industry methods that are known in the art. This vanillin may be provided in a solution, such as in an ethanolic solution.

The scent of vanilla may also be provided by ethylvanillin, a chemical compound that is related to vanillin. Ethylvanillin is approximately three times as potent as vanillin. The chemical structure of ethylvanillin, 3-ethoxy-4-hydroxybenzaldehyde, is shown below:

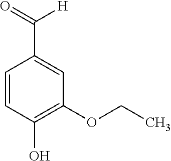

As used herein, the term vanillin may be used interchangeably with ethylvanillin.

As well as the scented substance itself, the food scent substance may contain additional ingredients. For example, carrier substances could be included to carry the food scent. These carrier substances may perform any function that enables the food scent to be carried, such as by modifying the volatility of the food scent. Such carrier substances would be well known to the skilled person. Alternatively, other ingredients may be included in the food scent substance such as emulsifying agents, stabilizing agents, binders, lubricants, buffers, diluents, preservatives and humectants. Suitable ingredients to perform these functions would be well known to the skilled person.

In one example of a vanilla food scent substance, the breakdown of ingredients by mass is as follows:

Natural Flavouring substances (including vanillin): 20.38%

Propylene Glycol (Carrier): 16.16%

Triacetin (Carrier): 63.46%

A total mass of between 200 milligrams and 1 gram of the food scent substance may be used inside an inhaler, for example between 400 milligrams and 800 milligrams, for example between 500 milligrams and 700 milligrams, for example between 550 milligrams and 650 milligrams. In one example, a total mass of 620 milligrams of the food scent substance is used inside an inhaler.

In use, a user removes the lid 104 from the body 102 and inserts the first end 106 of the body 102 into a nostril. The user then inhales the food scent from the food scent substance (e.g. intermittently) for a sufficient duration to suppress food cravings, for example at least two minutes. It will be understood that the exact quantity of food scent that has been inhaled by the user may be variable; different amounts of the food scent might be inhaled by different users. However, importantly, the inhaler allows the user to inhale the food scent substrate over a time period sufficient to suppress cravings for indulgent foods, for example at least two minutes. The inhaler 10 (particularly the body 102) can be applied to one or both nostrils according to the user's preference. Once inhalation has been completed, the user replaces the lid 104 on the body 102 and then inhaler 10 can then be stored conveniently (e.g. in a bag or pocket) until use is required again. Inhaler 10 contains an amount of the food scent substrate sufficient for repeat inhalation. Inhalation can be repeated as often as is required by the user. For example, inhalation may be repeated whenever the user has a craving for the food scent. The user may also inhale the food scent substrate at regular intervals throughout the day.

In one embodiment, the body 102 and the lid 104 are constructed from polypropylene. The skilled person will understand that any other suitable material (e.g. plastic) could be used. Additionally, the body 102 and the lid 104 could be 3D printed or constructed using additive layer manufacturing.

The present invention has been described above in exemplary form with reference to the accompanying drawings which represent a single embodiment of the invention. It will be understood that many different embodiments of the invention exist, and that these embodiments all fall within the scope of the invention as defined by the following claims.

1. Food scent substance for use in the treatment of obesity, wherein food scent from the food scent substance is inhaled from a device comprising the food scent substance.

2. The food scent substance for use of claim 1, wherein the device is at least partially inserted into a user's nostril whilst the food scent is being inhaled.

3. The food scent substance for use of claim 1 or claim 2, wherein the food scent is inhaled for at least two minutes.

4. The food scent substance for use of any one of the preceding claims, wherein the device comprises a device body, and wherein at least a portion of the device body has a substantially circumferential cross-sectional shape.

5. The food scent substance for use of any one of the preceding claims, wherein the device body has a first end that is inserted into a user's nostril and an opposing second end, wherein the first end has a rounded shape, for example a hemispherical shape.

6. The food scent substance for use of any one of the preceding claims, wherein the food scent is an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread.

7. The food scent substance for use of any one of the preceding claims, wherein the device contains a substrate and wherein the food scent substance is disposed on the substrate.

8. The food scent substance for use of claim 7, wherein the substrate comprises cotton wool.

9. A method comprising:
providing a device comprising a food scent substance; and
inhaling food scent from the food scent substance.

10. The method of claim 9, wherein the device is at least partially inserted into a user's nostril whilst the food scent is being inhaled.

11. The method of claim 9 or claim 10, wherein the food scent is inhaled for at least two minutes.

12. The method of any one of claims 9 to 11, wherein the device comprises a device body, and wherein at least a portion of the device body has a substantially circumferential cross-sectional shape.

13. The method of any one of claims 9 to 12, wherein the device body has a first end that is inserted into a user's nostril and an opposing second end, wherein the first end has a rounded shape, for example a hemispherical shape.

14. The method of any one of claims 9 to 13, wherein the food scent is an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread.

15. The method of any one of claims 9 to 14, wherein the device contains a substrate and wherein the food scent substance is disposed on the substrate.

16. The method of claim 15, wherein the substrate comprises cotton wool.

17. An inhaler comprising a food scent substance and means for inhaling a food scent from the food scent substance.

18. The inhaler of claim 17, wherein the inhaler comprises a body, and wherein at least a portion of the body has a substantially circumferential cross-sectional shape, and optionally:
wherein the device body has a first end that is configured to be inserted into a user's nostril and an opposing second end, wherein the first end has a rounded shape, for example a hemispherical shape.

19. The inhaler of claim 17 or claim 18, wherein the food scent is an indulgent food scent, such as vanilla, cookie, pizza, chocolate, ice cream, cake, biscuits, cupcakes, pastries, donuts, brownies, candy, sweets, popcorn, fizzy drinks, chips/fries, fried chicken, bacon, burgers or bread.

20. The inhaler of any one of claims 17 to 19, wherein the device contains a substrate and wherein the food scent substance is disposed on the substrate.
